(19) United States
(12) Patent Application Publication
(10) Pub. No.: US 2025/0355068 A1
(43) Pub. Date: Nov. 20, 2025

(54) Method and System for Determining an Orientation and a Position of a Movable Object Relative to a B0 Field Magnet

(71) Applicant: Siemens Healthineers AG, Forchheim (DE)

(72) Inventor: Johann Sukkau, Herzogenaurach (DE)

(73) Assignee: Siemens Healthineers AG, Forchheim (DE)

(21) Appl. No.: 19/209,948

(22) Filed: May 16, 2025

(30) Foreign Application Priority Data
May 16, 2024 (EP) 24176304.4

(51) Int. Cl. G01R 33/30; A61B 5/00; A61B 5/055; G01R 33/02

(52) U.S. Cl. CPC G01R 33/307; A61B 5/055; A61B 5/704; G01R 33/0206

(57) ABSTRACT

A method for determining the orientation and position of a movable object relative to the B0 field magnet of a magnetic resonance tomography (MRT) device in the X-Z plane of an X-Y-Z coordinate system aligned with the B0 field, may include: providing B0 reference data representing magnetic field strengths at multiple X-Y-Z coordinates; employing at least three three-dimensional magnetic field sensors fixed in known positions on the object; acquiring position data for each sensor by evaluating magnetic field measurement components independent of the object's orientation in the X-Z plane; filtering the sensor position data based on their known relative positions to yield filtered position data; and determining the object's orientation and position relative to the B0 field magnet using the filtered data. This technique allows precise localization and tracking within the magnetic field environment of the MRT system.

### CROSS REFERENCE TO RELATED APPLICATIONS

This patent application claims priority to European Patent Application No. 24176304.4, filed May 16, 2024, which is incorporated herein by reference in its entirety.

### BACKGROUND

### Field

The present disclosure relates to a method and a system for determining an orientation and a position of a movable object relative to a B0 field magnet of a magnetic resonance tomography device.

### Related Art

Magnetic resonance tomography devices are imaging apparatuses that, in order to image an examination object, align nuclear spins of the examination object with a strong external magnetic field and excite them to precession around this alignment using an alternating magnetic field. This precession or return of the spins from this excited state to a state with lower energy, in turn, generates in response an alternating magnetic field which is received via antennas.

With the aid of magnetic gradient fields, a spatial encoding is impressed upon the signals, which subsequently enables the received signal to be allocated to a volume element. The received signal is then evaluated, and a three-dimensional imaging representation of the examination object is provided.

Typically, the spatial encoding is based on an X-Y-Z coordinate system. The Z-coordinate axis is usually defined as an axis of symmetry of the B0 field magnet through a patient tunnel of the B0 field magnet in the direction of the B0 field. In the usual configuration of a magnetic resonance tomography device, the Z-coordinate axis is horizontally oriented and runs centrally through the opening of the windings of the B0 field magnet through a scanning area of the B0 field magnet. The object to be scanned is usually moved into the patient tunnel on a patient table parallel to the Z-coordinate axis. Together with a Z-coordinate axis, an X-coordinate axis and a Y-coordinate axis span a space, wherein the coordinate axes may be orthogonal to one another and the X-coordinate axis is horizontally oriented and the Y-coordinate axis is vertically oriented.

Typically, a person manually moves a mobile patient table close to the B0 field magnet, where the patient table is drawn using an automated gripping apparatus into a predetermined position at the B0 field magnet. The patient table, and consequently the patient, can then be subjected to sometimes strong shaking movements since an imprecise positioning or orientation of the patient table by the gripping apparatus must be corrected.

In this context, it has been established that there is a need to provide a method and a system to be able to provide an orientation and a position of a movable object relative to a B0 field magnet of a magnetic resonance tomography device. In particular, there is a need to be able to simplify the moving of a patient table toward the B0 field magnet.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 shows a plan view of a system according to an exemplary embodiment of the disclosure, in the form of a magnetic resonance tomography device.

FIG. 2 shows a lateral view of the magnetic resonance tomography device shown in FIG. 1.

FIG. 3 shows an arrangement of three magnetic field strength sensors relative to one another according to an exemplary embodiment of the disclosure.

FIG. 4 is a flowchart of a method for determining an orientation and a position of a movable object relative to a B0 field magnet of a magnetic resonance tomography device in an X-Z coordinate plane in an X-Y-Z coordinate system directed at the B0 field magnet, according to an exemplary embodiment of the disclosure.

FIG. 5 shows a schematic representation of the measurement value portions of a magnetic field strength sensor, which are independent of its orientation, according to an exemplary embodiment of the disclosure.

FIG. 6 shows a schematic representation of the point clouds of two magnetic field strength sensors, according to an exemplary embodiment of the disclosure.

FIG. 7 shows a schematic representation of the point clouds of three magnetic field strength sensors, according to an exemplary embodiment of the disclosure.

The exemplary embodiments of the present disclosure will be described with reference to the accompanying drawings. Elements, features and components that are functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software, or a combination of hardware and software.

An object of the present disclosure is to provide a solution with which the orientation and position of a movable object relative to a B0 field magnet of a magnetic resonance tomography device can be provided. In particular, it is an object of the present disclosure to simplify the moving of a patient table toward the B0 field magnet.

In accordance with the disclosure, a method is proposed for determining an orientation and a position of a movable object relative to a B0 field magnet of a magnetic resonance tomography device in an X-Z coordinate plane in an X-Y-Z coordinate system directed at the B0 field magnet, wherein the method may comprise at least the following steps:
a. providing B0 reference data of the B0 field magnet with characteristic magnetic field strengths for a multiplicity of X-Y-Z coordinates;
b. providing at least three three-dimensional magnetic field strength sensors which are arranged in a fixed relative position on the object;
c. ascertaining position data for a respective magnetic field strength sensor by evaluating measurement value portions of the respective magnetic field strength sensor, the measurement value portions being independent of the respective orientation of the movable object in the X-Z coordinate plane;
d. filtering the ascertained position data of the magnetic field strength sensors based on the fixed relative position of the magnetic field strength sensors and providing filtered position data; and
e. providing an orientation value and a position of the movable object relative to the B0 field magnet based on the filtered position data.

In other words, the present disclosure proposes, in a first step, to ascertain the position data for the respective magnetic field strength sensors based on measurement value portions of the magnetic field strength sensors, the measurement value portions being independent of an orientation of the magnetic field strength sensors in the X-Z coordinate plane.

The position data ascertained in this manner does not, in principle, result in unique point coordinates, but in point data with a multiplicity of possible point coordinates which are also referred to as a point cloud. The position data ascertained in this manner is filtered in a further step with the aid of the known fixed relative positions of the magnetic field strength sensors with respect to one another. In the present context, filtering is to be understood to mean selecting from the ascertained position data/point data those points or point coordinates which can be brought into correlation with the known relative positions of the magnetic field strength sensors. In this regard, the ascertained position data can be filtered with the aid of different conditions which arise from the known relative positions of the magnetic field strength sensors with respect to one another. This filtered position data can be subsequently used in order to be able to ascertain with a sufficiently high degree of accuracy an orientation value, for example in the form of an angle of the object relative to the B0 field magnet, and a position of the object, for example in the form of an X-Z coordinate. This orientation value and the position of the object can be subsequently used, for example, in order to ascertain control data for a navigation and movement facility of the object.

Fundamentally, in the present case, two X-Y-Z coordinate systems or reference systems can differ. On the one hand, a reference system which is directed at the B0 field magnets. In this reference system, the Z-coordinate axis corresponds to an axis of symmetry of the B0 field magnet in the direction of the B0 field, wherein the coordinate axes may be provided orthogonally to one another and the X-coordinate axis may be horizontally oriented and the Y-coordinate axis may be vertically oriented. Such a coordinate system that is directed at the B0 field magnets is also referred to in practice as a so-called “device coordinate system” (DCS).

A further X-Y-Z coordinate system can be directed at the object. If the object is, for example, a patient table, the zero point of the X-Y-Z coordinate system can be provided centrally on the front edge of the patient table, the Z-coordinate axis can be oriented, for example, along the longitudinal axis of the patient table, the X-coordinate axis can be oriented horizontally and the Y-coordinate axis can be oriented vertically, wherein in turn the coordinate axes are provided orthogonal to one another. Such a coordinate system that is directed at a patient table is referred to in practice as a so-called “table coordinate system” (TCS).

The B0 reference data of the B0 field magnet are usually provided in the form of a 3D grid with a typical resolution of 1 mm, 5 mm or 1 . . . 10 mm. The B0 reference data includes an unambiguous allocation between a point in the space and the B0 vector in this point. The position of a point in the space, such as for example, a fixed point of the patient table, can be specified in the DCS coordinates by a vector p=(px, py, pz), wherein px, py, pz are the coordinates of the point in the respective space direction, specified for example in mm. The direction/orientation of a B0 field line in the X-Z plane, referred to, for example, as angle α, can be calculated via the atan2 function as follows: α=atan2(h.x, h.z).

In an exemplary embodiment, the at least three magnetic field strength sensors are arranged in the X-Z coordinate plane and thus have the same Y-coordinate.

In an exemplary embodiment, in an X-Y-Z coordinate system directed at the object at least two of the at least three magnetic field strength sensors have the same Z-coordinate, wherein, in one or more embodiments, at least two of the three magnetic field strength sensors also have the same X-coordinate.

In an exemplary embodiment, the measurement value portions independent of the orientation of the movable object include: the value of the B0 field vector (abs (h)), the Y-field strength components (h.y), Y-field strength components standardized to the value of the B0 field vectors (h.y/abs (h)) and/or the value of the vector in the X-Z coordinate plane (abs (h.x, h.z)), meaning the length of the vector (h.x, h.z).

In an exemplary embodiment, the step of filtering may comprise at least the following steps:
a. ascertaining point pairs from the ascertained position data from two magnetic field strength sensors, in which the distance corresponds to the fixed relative position of the two magnetic field strength sensors on the object;
b. ascertaining an orientation angle for the points of an ascertained point pair and ascertaining point pairs, in which the orientation angle for the points of the point pair are identical;
c. ascertaining point-pair combinations, in which the distance and the orientation of the point-pair combinations correspond to the fixed relative position of the at least three magnetic field strength sensors.

In an exemplary embodiment, the movable object is a patient table, wherein the magnetic field strength sensors may be arranged in the lower region at the side corner areas of the patient table.

In an exemplary embodiment, the method may further comprise: providing control data based on the provided orientation value and the position of the movable object for a navigation and movement facility which is configured to move the movable object to a target position relative to the B0 field magnet. In an exemplary embodiment, the control data is provided cyclically, wherein in addition at least one movement trajectory of the movable object and/or at least one direction vector and/or speed vector of the movable object are taken into consideration when providing the cyclic control data. For example, the position and the orientation of the patient table with respect to the B0 field magnet can be determined 50 to 100 times per second and, for example, made available to a navigation controller, so that as a result the patient table can be navigated and moved in an automated manner, in particular until the patient table docks with the B0 field magnet. In an exemplary embodiment, the position of the patient table is specified by a 2D vector, with X- and Z-coordinates in the DCS reference system. The orientation of the patient table may be specified by an angle (yaw) in the horizontal plane between the Z-coordinate axis of the B0 field magnet and the longitudinal direction of the patient table. Consequently, an X-coordinate, a Z-coordinate and the angle (yaw) of the patient table are transmitted to the navigation and movement facility approx. 50 to 100 times per second so that as a result the patient table can be reliably moved in the B0 stray field.

In an exemplary embodiment, the method nay further comprise: providing at least one collision sensor which is arranged on the object and is configured to scan an area around the object and to ascertain whether objects are located in a planned movement path, wherein at least three collision sensors may be provided on the object, and wherein the collision sensor or collision sensors (160) may scan an area with an opening angle of at least 90°, preferably of at least 180° and particularly preferably of at least 270°. It is possible to use different collision sensors which, for example, cover an area in front of the patient table, to the side of the patient table. By way of example, it is also possible to use a LIDAR sensor here.

As already mentioned, the B0 field magnet may enclose a patient tunnel of the magnetic resonance tomography device, wherein the Z-coordinate axis is defined by an axis of symmetry of the B0 field magnet in the direction of the B0 field, wherein the coordinate axes may be provided orthogonal to one another and the X-coordinate axis may be horizontally oriented and the Y-coordinate axis may be vertically oriented.

In an exemplary embodiment, a magnetic field strength sensor is configured to detect a field strength of three components of the B0 field in three directions, which span a space.

Furthermore, the present disclosure relates to a system for determining an orientation and a position of a movable object relative to a B0 field magnet of a magnetic resonance tomography device in an X-Z coordinate plane in an X-Y-Z coordinate system directed at the B0 field magnet. The system may comprise:
a. a first interface configured to receive B0 reference data of the B0 field magnet with characteristic magnetic field strengths for a multiplicity of X-Y-Z coordinates;
b. a second interface configured to receive measurement values of the at least three magnetic field strength sensors; and
c. a computing unit (computer, processor) which is connected to the interfaces and is configured to perform the above described method.

Furthermore, the present disclosure relates to a use of a movable object with at least three three-dimensional magnetic field strength sensors in an above-described method. The movable object may be an above-described patient table.

Furthermore, the present disclosure relates to a computer program element with instructions which when executed on data-processing devices of a data processing environment are configured to perform the steps of the above-mentioned method in an above-mentioned system.

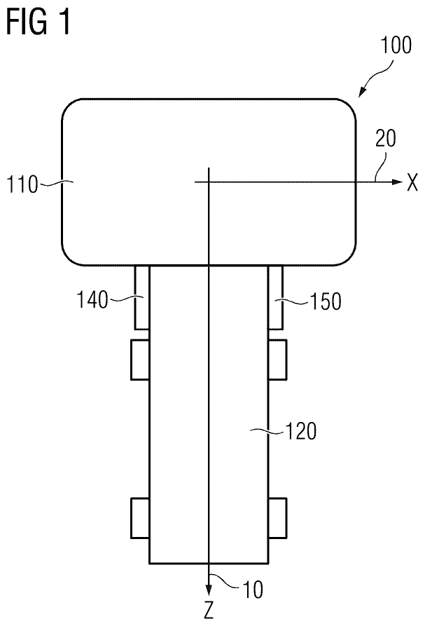

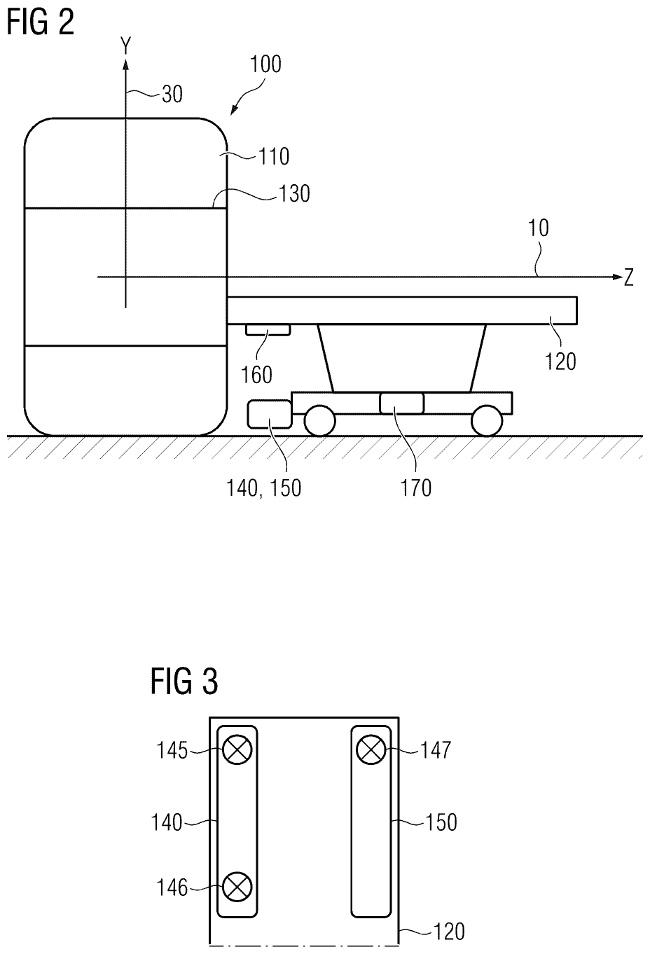

FIG. 1 shows a plan view and FIG. 2 shows a lateral view of a system 100 in accordance with the disclosure, in the form of a magnetic resonance tomography device 100. The magnetic resonance tomography device 100 may comprise a B0 field magnet 110 (referred to as a scanner) and a patient table 120.

As shown in FIGS. 1 and 2, the spatial encoding is based on an X-Y-Z coordinate system which is directed at the B0 field magnet, a so-called DCS reference system. The Z-coordinate axis 10 is usually defined as an axis of symmetry of the B0 field magnet 110 through a patient tunnel 130 of the B0 field magnet 110 in the direction of the B0 field. The Z-coordinate axis 10 is horizontally oriented in the case of the illustrated, usual arrangement of the magnetic resonance tomography device 100 and runs centrally through the opening of the windings of the B0 field magnet 110 through the patient tunnel 130 of the B0 field magnet 110. The object to be scanned is usually moved into the patient tunnel 130 on the patient table 120 parallel to the Z-coordinate axis 10. Together with the Z-coordinate axis 10, an X-coordinate axis 20 and a Y-coordinate axis 30 span a space, wherein the X-Y-Z coordinate axes may be provided orthogonally to one another and wherein the X-coordinate axis is horizontally oriented and the Y-coordinate axis is vertically oriented. The patient table 120 also comprises a first magnetic field strength sensor unit 140, a second magnetic field strength sensor unit 150, a collision sensor unit 160 and an evaluation and navigation unit 170. The evaluation and navigation unit 170 may also be referred to as a computing unit, computer, or controller. The evaluation and navigation unit 170 may comprise processing circuitry that is configured to perform on or more functions and/or operations of the evaluation and navigation unit 170. The evaluation and navigation unit 170 may include one or more memory units (memory) that is adapted to store one or more computer programs and/or data. As shown in FIG. 3, the first magnetic field strength sensor unit 140 in the illustrated exemplary embodiment comprises two three-dimensional magnetic field strength sensors 145, 146 and the second magnetic field strength sensor unit 150 comprises a magnetic field strength sensor 147. The distances and positioning of the magnetic field strength sensors 145, 146, 147 relative to one another are known.

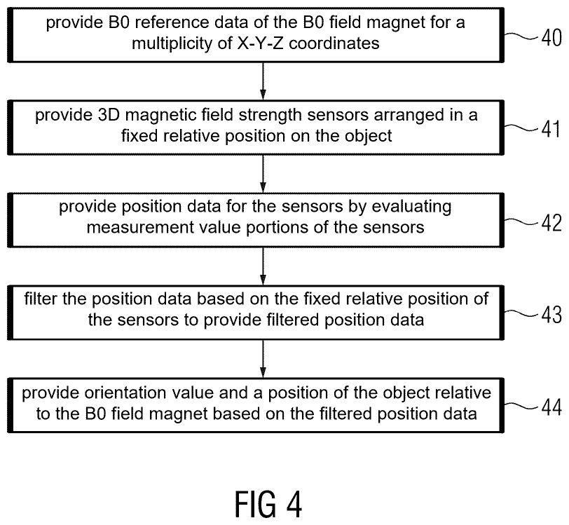

FIG. 4 shows a schematic representation of a method in accordance with the disclosure for determining the orientation and the position of the movable object 120 relative to the B0 field magnet 110 of the magnetic resonance tomography device 100 in the X-Z coordinate plane in the DCS reference system.

In a first step 40, B0 reference data of the B0 field magnet 110 with characteristic magnetic field strengths for a multiplicity of X-Y-Z coordinates is determined or otherwise provided. In a further step 41, at least three three-dimensional magnetic field strength sensors 145, 146, 147 are provided, which are arranged in a fixed relative position on the object 120. In a further step 42, position data for the respective magnetic field strength sensors 145, 146, 147 is determined or otherwise provided by evaluating measurement value portions of the respective magnetic field strength sensors 145, 146, 147, the measurement value portions being independent of the respective orientation of the movable object 120 in the X-Z coordinate plane. In a further step 43, the ascertained position data of the magnetic field strength sensors 145, 146, 147 is ascertained based on the fixed relative position of the magnetic field strength sensors 145, 146, 147 and filtered position data provided. Finally, in a step 44, an orientation value and a position of the movable object 120 relative to the B0 field magnet 110 are determined (or otherwise provided) based on the filtered position data.

Exemplary embodiments of a method and a system are described below. In one or more exemplary embodiments, the orientation value and the position of the movable object 120 is used to provide control data for a navigation and movement facility of the object 120, in this case in the form of an autonomously drivable patient table 120.

In one or more exemplary embodiments, the patient table 120 may comprise at least three three-dimensional magnetic field strength sensors 145, 146, 147. The measurement data of the magnetic field strength sensors 145, 146, 147 can be transmitted to the evaluation and navigation unit (controller) 170 and processed by this evaluation and navigation unit 170 into control data. The control data can be used to control the patient table 120 and/or the magnetic resonance tomography device 100 (e.g., scanner). The measurement data can be combined in the evaluation and navigation unit 170, and, by applying the method in accordance with the disclosure, it is possible to provide orientation values and the position of the patient table 120 relative to the B0 field magnets 110. In an exemplary embodiment, an X-coordinate, a Z-coordinate, and the angle (yaw) of the patient table 120 are ascertained approximately 50 to 100 times per second. The evaluation and navigation unit 170 can subsequently determine the next path point on the path on which the patient table 120 moves to dock with the B0 field magnet 110 and transmit corresponding control commands, for example, to servomotors for moving and steering the patient table 120. In addition, data from the collision sensor unit 160 can be transmitted to the evaluation and navigation unit 170 in order to ensure that there are no objects/persons located in the travel path of the patient table 120.

In an exemplary embodiment, the magnetic field strength sensor units 140, 150, and consequently the magnetic field strength sensors 145, 146, 147 arranged therein are arranged in the lower region on the patient table 120, as close as possible to the floor. In this case, in an exemplary embodiment, the magnetic field strength sensor units 140, 150 are arranged on the side of the patient table 120. It is thus possible to achieve that the B0 magnetic field, which is measured by the magnetic field strength sensors 145, 146, 147, is also comparatively small in the immediate vicinity of the B0 field magnet 110, so that the B0 magnetic field can also be measured using cost-effective magnetic field strength sensors. Furthermore, it is also possible using the arrangement of the magnetic field strength sensor units 140, 150 to ensure that no large metal parts of the patient table 120 are provided in the vicinity of the magnetic field strength sensors 145, 146, 147, and that the B0 magnetic field, which is measured by the magnetic field strength sensors 145, 146, 147 is not disrupted.

As mentioned, the position data for the magnetic field strength sensors 145, 146, 147 is first ascertained by evaluating measurement value portions of the respective magnetic field strength sensors 145, 146, 147, the measurement value portions being independent of the respective orientation of the movable object in the X-Z coordinate plane. The values h.x and h.z which are dependent on the orientation of a magnetic field strength sensor 145, 146, 147 are not used here. The respective measurement value portions are represented in FIG. 5. The average quantity of the measurement value portions represents the position data/point clouds of a magnetic field strength sensor 145, 146, 147. Fundamentally, the greater the point cloud, the further the magnetic field strength sensor 145, 146, 147 is from the B0 field magnet 110. The closer the magnetic field strength sensor 145, 146, 147 moves to the B0 field magnet 110, the smaller the point cloud. This breaks down into two or even four parts due to the rotational symmetry of the B0 field around the Z-coordinate axis.

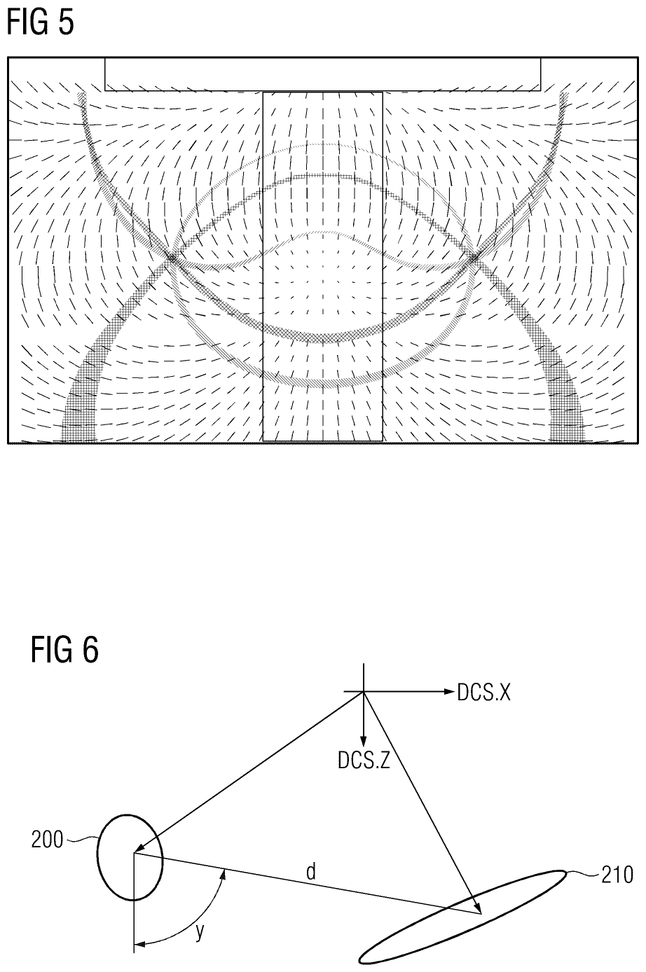

In a further step, the point clouds 200, 210 of two of the magnetic field strength sensors 145, 146, 147 are combined with one another. As illustrated in FIG. 6, those point pairs are ascertained/filtered out in which the distance d corresponds to the actual distance of the two magnetic field strength sensors 145, 146, 147 being considered. Corresponding point pairs are ascertained for the different two-part combinations of magnetic field strength sensors 145, 146, 147. In so doing, it is to be taken into consideration that point pairs are determined which correspond to the known distance d within a predetermined tolerance. For example, point pairs with a tolerance of +/−3%, +/−2% or +/−1% are formed from the respective value of the measurement portion. Alternatively, other tolerance limits/ranges can be applied.

In a further step, the above ascertained point pairs of two magnetic field strength sensors 145, 146, 147 are filtered with respect to their orientation. Due to the fixed installation of the magnetic field strength sensors 145, 146, 147 on the patient table 120, all the magnetic field strength sensors 145, 146, 147 have the same orientation, i.e. the same yaw angle. The orientation, represented in the form of the yaw angle, can be calculated at each ascertained point pair. This can be performed, for example, by means of the atan2 function, wherein α=atan2(h.x, h.z). Therefore, those point pairs in which the orientation of the magnetic field strength sensors 145, 146, 147 is identical are ascertained/filtered out from the above ascertained point pairs of two magnetic field strength sensors 145, 146, 147. In an exemplary embodiment, point pairs are ascertained which have the same orientation within a predetermined tolerance. For example, it is possible to filter point pairs which, with a tolerance of +/−3%, +/−2% or +/−1%, have an identical orientation. Alternatively, it is also possible to use other tolerance limits/ranges here.

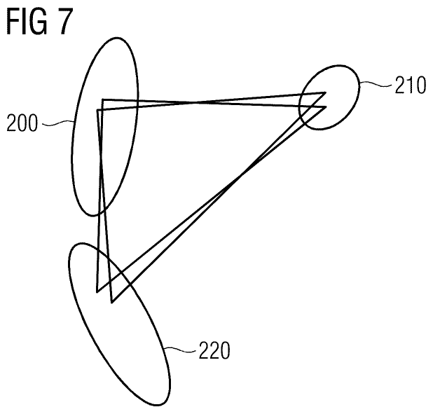

In a further step, the ascertained point pairs of the magnetic field strength sensors 145, 146, 147 with the same orientation are filtered once again by ascertaining those point pairs of the magnetic field strength sensors 145, 146, 147 that in combination correspond to the actual geometry of at least three magnetic field strength sensors 145, 146, 147. FIG. 7 shows by way of example two solutions of these point pair combinations which map the actual geometry of the at least three magnetic field strength sensors 145, 146, 147. Consequently, in one or more exemplary embodiments, those point pairs of the magnetic field strength sensors 145, 146, 147 are to be ascertained which correspond to the triangular arrangement shown in FIG. 3 of three magnetic field strength sensors 145, 146, 147. In an exemplary embodiment, corresponding tolerances may be predefined. For example, combinations can also be filtered here which, with a tolerance of +/−3%, +/−2% or +/−1%, correspond to the actual geometry of the at least three magnetic field strength sensors 145, 146, 147. Alternatively, it is also possible to use other tolerance limits/ranges here.

The remaining point pairs of the magnetic field strength sensors 145, 146, 147 are used for ascertaining a position of a magnetic field strength sensor 145, 146, 147. These positions of the magnetic field strength sensors 145, 146, 147 are subsequently used to ascertain in each case an X-coordinate, a Z-coordinate and an angle value of the patient table 120. A respective average value of the thus ascertained X-coordinates, Z-coordinates and angle values, can be subsequently used to provide control data for the navigation and movement facility of the patient table 120, in order to move the patient table 120 to a target position relative to the B0 field magnet 110. In an exemplary embodiment, the control data is provided cyclically, wherein, in one or more embodiments, in addition at least one movement trajectory of the patient table 120 and/or at least one direction vector and/or speed vector of the patient table 120 are taken into consideration when providing the cyclic control data. For example, the position and the orientation of the patient table 120 with regard to the B0 field magnet 110 can be determined 50 to 100 times per second and, for example, made available to a navigation controller, so that as a result the patient table 120 can be navigated and moved in an automated manner, in particular until the patient table 120 docks with the field magnet 110.

In an exemplary embodiment, the position of the patient table 120 is specified by a 2D vector, with X- and Z-coordinates in the DCS reference system. The orientation of the patient table may be specified by an angle (yaw) in the horizontal plane between the Z-coordinate axis of the B0 field magnet 110 and the longitudinal direction of the patient table 120. An X-coordinate, a Z-coordinate and the angle (yaw) of the patient table 120 may be transmitted to the navigation and movement facility approx. 50 to 100 times per second, so that as a result the patient table 120 can be reliably moved in the B0 magnetic field.

As a result, by virtue of the method in accordance with the disclosure or the system in accordance with the disclosure, a patient table 120 can be docked with a B0 field magnet 110 in an automated and gentle manner, which considerably increases patient comfort. In addition, the automated docking can be performed comparatively quickly, in particular in comparison to the patient table 120 being moved manually by an operator. The method in accordance with the disclosure or the system in accordance with the disclosure can also be used independently for other applications if a position and an orientation of an object relative to a B0 field magnet is to be provided reliably, precisely and quickly.

The present disclosure is not limited to the above described embodiment, provided that it is covered by the subject matter of the claims below. In particular, the present disclosure is not limited to the use of the three magnetic field strength sensors 145, 146, 147. It is possible by using further magnetic field strength sensors to provide advantageous redundancies, a greater degree of accuracy and reliability and an immunity to measurement noises. In an exemplary embodiment, the magnetic field strength sensor units each comprise up to 12 magnetic field strength sensors, so that when using three magnetic field strength sensor units a total of 36 magnetic field strength sensors can be used.

In addition, it should be noted that the terms “comprising” and “having” do not exclude other elements or steps and the indefinite articles “a” or “an” do not exclude a plurality. Furthermore, it should be noted that features or steps described with reference to the above embodiments can also be used in combination with other features.

Furthermore, it should be noted that independent of the grammatical term usage, individuals with male, female or other gender identities are included within the term.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

The various components described herein may be referred to as “modules,” “units,” or “devices.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such modules, units, or devices, as applicable and relevant, may alternatively be referred to herein as “circuitry,” “controllers,” “processors,” or “processing circuitry,” or alternatively as noted herein.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A method for determining an orientation and a position of a movable object relative to a B0 field magnet of a magnetic resonance tomography device in an X-Z coordinate plane in an X-Y-Z coordinate system directed at the B0 field magnet, the method comprising:
providing B0 reference data of the B0 field magnet with characteristic magnetic field strengths for a multiplicity of X-Y-Z coordinates;
providing at least three three-dimensional (3D) magnetic field strength sensors which are arranged in a fixed relative position on the movable object;
ascertaining position data for a respective one of the at least three 3D magnetic field strength sensors by evaluating measurement value portions of the respective magnetic field strength sensor, the measurement value portions being independent of the respective orientation of the movable object in the X-Z coordinate plane;
filtering the ascertained position data of the at least three 3D magnetic field strength sensors based on the fixed relative position of the at least three 3D magnetic field strength sensors and providing filtered position data; and
providing, based on the filtered position data, an orientation value and a position of the movable object relative to the B0 field magnet in electronic form as an output data file.

2. The method as claimed in claim 1, wherein the at least three 3D magnetic field strength sensors are arranged in the X-Z coordinate plane and have a same Y-coordinate.

3. The method as claimed in claim 1, wherein, in an X-Y-Z coordinate system directed at the movable object, at least two of the at least three 3D magnetic field strength sensors have a same Z-coordinate and at least two of the three 3D magnetic field strength sensors have a same X-coordinate.

4. The method as claimed in claim 1, wherein the measurement value portions, independent of the orientation of the movable object, comprise: a value of the B0 field vector (abs (h)), the Y-field strength components (h.y), Y-field strength components standardized to the value of the B0 field vectors (h.y/abs (h)), and/or a value of the vector in the X-Z coordinate plane (abs (h.x, h.z)).

5. The method as claimed in claim 1, wherein the position data is provided as point data or point clouds.

6. The method as claimed in claim 1, wherein the filtering comprises:
ascertaining point pairs from the ascertained position data from two of the at least three 3D magnetic field strength sensors, in which a distance corresponds to the fixed relative position of the two magnetic field strength sensors on the object;
ascertaining an orientation angle for points of an ascertained point pair, in which the orientation angle for the points of the point pair are identical;
ascertaining point-pair combinations, in which a distance and an orientation of the point-pair combinations correspond to the fixed relative position of the at least three 3D magnetic field strength sensors.

7. The method as claimed in claim 1, wherein the movable object is a patient table, and wherein the at least three 3D magnetic field strength sensors are arranged in a lower region at a side corner areas of the patient table.

8. The method as claimed in claim 1, further comprising: providing control data, by a controller and based on the provided orientation value and the position of the movable object, to control the movable object to move to a target position relative to the B0 field magnet.

9. The method as claimed in claim 8, wherein the control data is provided cyclically, at least one movement trajectory of the movable object and/or at least one direction vector and/or speed vector of the movable object are considered when providing the cyclic control data.

10. The method as claimed in claim 8, further comprising: providing at least one collision sensor arranged on the object and that is configured to scan an area around the object and to ascertain whether objects are located in a planned movement path.

11. The method as claimed in claim 10, wherein the collision sensor is configured to scan an area with an opening angle of at least 90°.

12. The method as claimed in claim 1, wherein the B0 field magnet encloses a patient tunnel of the magnetic resonance tomography device, wherein the Z-coordinate axis is defined by an axis of symmetry of the B0 field magnet in the preferred direction of the B0 field, the coordinate axes being provided orthogonal to one another and the X-coordinate axis being horizontally oriented and the Y-coordinate axis being vertically oriented.

13. The method as claimed in claim 1, wherein the at least three 3D magnetic field strength sensors are configured to detect a field strength of three components of the B0 field in three directions spanning a space.

14. One or more non-transitory media storing instructions that, when executed by one or more processors, cause the one or more processors to perform the method of claim 1.

15. A system for determining an orientation and a position of a movable object relative to a B0 field magnet of a magnetic resonance tomography device in an X-Z coordinate plane in an X-Y-Z coordinate system directed at the B0 field magnet, the system comprising:
a first interface configured to receive B0 reference data of the B0 field magnet with characteristic magnetic field strengths for a multiplicity of X-Y-Z coordinates;
a second interface configured to receive measurement values of the at least three magnetic field strength sensors; and
a controller connected to the first and the second interfaces, the controller being configured to perform the method as claimed in claim 1.

16. An apparatus comprising:
one or more processors; and
memory storing instructions that, when executed by the one or more processors, cause the apparatus to:
provide B0 reference data of the B0 field magnet with characteristic magnetic field strengths for a multiplicity of X-Y-Z coordinates;
provide at least three three-dimensional (3D) magnetic field strength sensors which are arranged in a fixed relative position on the movable object;
ascertain position data for a respective one of the at least three 3D magnetic field strength sensors by evaluating measurement value portions of the respective magnetic field strength sensor, the measurement value portions being independent of the respective orientation of the movable object in the X-Z coordinate plane;
filter the ascertained position data of the at least three 3D magnetic field strength sensors based on the fixed relative position of the at least three 3D magnetic field strength sensors and providing filtered position data; and
provide, based on the filtered position data, an orientation value and a position of the movable object relative to the B0 field magnet in electronic form as an output data file.
